(19) United States
(12) Patent Application Publication
(10) Pub. No.: US 2007/0213838 A1
(43) Pub. Date: Sep. 13, 2007

(54) Tubular Graft

(75) Inventor: Rudy Hengelmolen, London (GB)

Correspondence Address: ROTHWELL, FIGG, ERNST & MANBECK, P.C., 1425 K STREET, N.W., SUITE 800, WASHINGTON, DC, 20005, US

(73) Assignee: Auxetica Limited, London (GB)

(21) Appl. No.: 10/588,158

(22) PCT Filed: Feb. 2, 2005
(86) PCT No.: PCT/GB05/00365

§ 371 Date: May 18, 2007

(30) Foreign Application Priority Data
Feb. 2, 2004 (GB) 0402103.6

(51) Int. Cl. A61F 2/04; A61F 2/84; A61F 2/90

(52) U.S. Cl. 623/23.71; 623/1.16; 606/108; 216/8

(57) ABSTRACT

The present invention is concerned with a tubular graft comprising a first auxetic tube (20D) defining an interior surface and an exterior surface, and having a non-auxetic tubular covering (20A) on at least one of the group consisting of: said exterior surface, and said interior surface. Also provided are methods of manufacture of same and methods of use of same.

The present invention is concerned with tubular grafts, particularly prosthetic tubular grafts, comprising an auxetic tube with a non-auxetic tubular covering, together with methods of manufacture of same, and methods of use of same. The grafts of the present invention can be used in a wide range of applications and to replace a wide range of in vivo ducts such as blood vessels, bile ducts in the liver or pancreas, gastrointestinal tubes such as the oesophagus, urethra and ureter ducts and pulmonary passageways, and can also be used as stent grafts (e.g. to support an existing section of duct).

According to the present invention there is provided a tubular graft comprising a first auxetic tube defining an interior surface and an exterior surface, and having a non-auxetic tubular covering on at least one of the group consisting of: said interior surface, and said exterior surface.

The at least one tubular covering may be contiguous with the first auxetic tube.

The first auxetic tube may define first and second ends and a lumen, both of said first and second ends being open, such that fluid flow can occur through the first auxetic tube from the first end to the second end.

References to auxetic material herein include materials which are intrinsically auxetic and materials which have been rendered auxetic (as discussed hereinafter).

The at least one tubular covering may be impermeable. Alternatively, the at least one tubular covering may be permeable to a desired extent. For example, one or more tubular coverings may have been made semi-permeable by processing or by chemical treatment e.g. by plasmic treatment using H₂. The at least one covering may also be treated (for example, chemically treated) in order to effect surface porosity, leaving any material which is contiguous with the first auxetic tube relatively impermeable.

n the case of a graf comprising a first auxetic tube and a non-auxetic exterior covering, he first auxetic tube and the exterior covering may be physically distinct from one another, and may be held together by ionic forces at their interface, e.g. friction caused between the two may cause resistance to their movement against one another, hence keeping them together. In order to manufacture such a graft, an auxetic first tube may be compressed such that it has an outer diameter less than the interior diameter of the non-auxetic exterior covering, and may then be inserted into the lumen of the exterior covering and allowed to expand, making the two contiguous and forming a graft.

In the case of a graft comprising a first auxetic tube and a non-auxetic interior covering, this may be manufactured as a single tube. For example, it may be fabricated from a single material (e.g. a polymer) or it may consist at least two co-extruded layers, each contiguous pair of layers being made of different polymers. In this case, the first auxetic tube extends part way into the non-auxetic covering (for example at least 1, 2, 3, 4, 5, 6, 7, 8, 9, 10, 15, 20, 25, 30, 40 or 50% of the thickness of the wall of the interior covering). This arrangement can be readily achieved using controlled laser treatment. The extension of the material of the first auxetic tube into a co-extruded interior covering need not stop at a specific interface between the two. In such cases, the first auxetic tube and the covering tend to be held together more strongly than in the first case (above) where an auxetic tube expands and contacts a non-auxetic exterior covering. The first auxetic tube and non-auxetic interior covering may be held together by ionic forces, and may also be held together by covalent bonding for example due to bonding occurring during a co-extrusion process or during laser treatment.

Other thermal treatments may be used to effect bonding between the first auxetic tube and the at least one covering. Alternatively, adhesives may be used to effect bonding between them, although of course the use of any such adhesive must not interfere with the auxetic properties of the first auxetic tube. Known adhesives include e.g. thermoplastic fluoropolymers such as fluorinated ethylene propylene (FEP) which can be used to effect bonding by heat-treatment. Suitable adhesives and the conditions for their use will be readily apparent to a person skilled in the art.

In the grafts of the present invention, the first auxetic tube acts as a scaffold to provide support for the non-auxetic at least one covering, the non-auxetic at least one covering being relatively thin (for example having a thickness no greater than 5, 10, 15, 20, 25, 30, 40 or 50% the thickness of the first auxetic tube). The non-auxetic at least one covering thus serves to control or prevent lateral permeability, resulting in a structure which overall will still be auxetic in response to outside mechanical stimuli.

In cases where the graft comprises a first auxetic tube with both exterior and interior non-auxetic tubular coverings, the graft may be manufactured by first making a graft comprising a first auxetic tube and a non-auxetic inner covering, for example by co-extrusion as detailed above. The first auxetic tube and inner non-auxetic covering can then be compressed, inserted into the lumen of a non-auxetic exterior tubular covering, and then allowed to expand so that the exterior covering is contiguous with the first auxetic tube.

As an alternative to the use of a pre-fabricated non-auxetic tube as an exterior covering, a non-auxetic exterior covering can also be fabricated around the first auxetic tube, for example by spinning threads, particularly polymer threads, around the first auxetic tube so as to form a non-auxetic exterior covering. Such a non-auxetic exterior covering may be permeable.

Thus the present invention provides a robust auxetic scaffold, particularly using the auxetic structures described below, combined with at least one relatively thin, non-auxetic covering layer, which retains the auxetic integrity in the overall structure in response to mechanical stimuli, e.g. from the exerted by the environment of the graft. In this way, a graft is provided whose non-auxetic part(s) can be manipulated such that it possesses selective permeability in order to e.g. stimulate endothelial cell attachment or to control and target drug delivery from the graft.

The prior art does not provide any suggestion of the construction of the grafts of the present invention with their combination of auxetic and non-auxetic properties and their usefulness and ability to stimulate endothelial cell attachment and effect drug delivery. In particular, the non-auxetic materials do not hinder or have any significantly adverse effect upon the auxetic properties of the first auxetic tube.

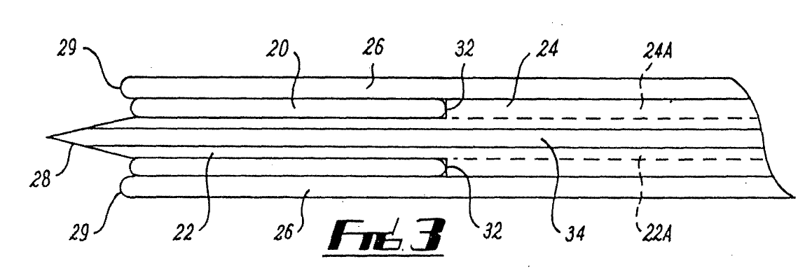

As mentioned above, the present invention makes use of auxetic materials. Conventional materials have a positive Poisson ratio, i.e. when stretched in one direction they tend to become thinner in a direction lateral to the direction of elongation—Poisson's ratio is the ratio of the lateral contraction per unit breadth, to the longitudinal extension per unit length when a piece of material is stretched. Other materials are designed such that they have a Poisson ratio of zero. For example, a tube may be designed such that it is radially expansible and compressible without any longitudinal change in size. Auxetic materials exhibit a negative Poisson ratio in that they expand in a direction perpendicular to the direction of stretching. Auxetic materials also have the capacity for formation into doubly curved or dome shaped surfaces due to the synclastic property of auxetic materials, a property which is described in for instance WO 99/22838 with reference to FIG. 2(b) thereof.

Thus with the tubular grafts of the present invention, when they are radially compressed, they become shorter, whereas when they are radially expanded, they increase in length.

The auxetic material may be a synthetic auxetic material and may have a macroscopic or microscopic auxetic structure.

The auxetic material may be polymeric.

The first auxetic tube may be in the form of a metallic, auxetic mesh structure.

The auxetic material may be of a porous nature.

The auxetic material of the first auxetic tube may comprise a biodegradable polymer or polymers, useful in situations where it is desirable for the auxetic tube structure to degrade over time. For example, it may be that the auxetic tube has a non-auxetic tubular covering over its interior surface which promotes cell growth and adhesion, thus the graft initially acting as a graft to join two sections of duct (e.g. blood vessel), and subsequently degrading over time whilst promoting cellular growth over the non-auxetic tubular covering(s). In other situations it may be that a permanent graft is required, in which case the first auxetic tube may not be biodegradable.

Examples of biodegradable polymers include polyglycolic acid and its copolymers, polylactic acid (both D and L isomers) and their copolymers, poly-β-hydroxybutyrate, poly-β-hydroxypropionate, poly-ε-caprolactone, poly-δ-valerolactone, poly(methylmethacrlate-co-N-vinylpyrrolidone), polyvinyl alcohol, polyanhydrides, poly-ortho-esters, and polyphosphazenes. Of particular use are polyglycolic acid (PGA), as well as its copolymers and the isomeric polylactic acids, PLLA and PDLLA, together with their copolymers. Polymers and copolymers of E-caprolactone are also highly useful. Other biodegradable materials are detailed in: The Chemistry of Medical and Dental Materials, J W Nicholson, Royal Society of Chemistry, ISBN: 0854045724.

An auxetic material for use in the invention may be selected from any suitable material, including the known auxetic materials mentioned below.

Synthetic auxetic materials are known from for example U.S. Pat. No. 4,668,557 which discloses preparation of an open-celled polymeric foam, negative Poisson ratio properties being secured by mechanical deformation of the foam by compression. Auxetic materials may also be in the form of microporous polymers, polymer gels, and macroscopic cellular structures (e.g. structures comprising re-entrant “bow tie” or inverted hexagon units). A polymeric material is disclosed in WO 91/01210, the material having an auxetic microstructure of fibrils connected at nodes and being obtained by compacting polymer particles at elevated temperatures and pressures, sintering and then deforming the compacted polymer by extrusion through a die to produce a cylindrical rod of auxetic material. WO 00/53830 discloses an auxetic polymeric material which is of filamentary or fibrous form which is produced by cohering and extruding thermoformable particulate material, cohesion and extrusion being effected with spinning so that an auxetic microstructure of fibrils and nodes can be obtained without requiring separate sintering and compaction stages. Auxetic materials have for example been produced of polytetrafluoroethylene (PTFE), polyethylene, nylon and polypropylene. Particularly useful materials for the auxetic tubular liners of the present invention are nylon, polyurethanes and polyesters.

Other materials include collagens and collagen-based materials such as those of U.S. Pat. No. 5,162,430 and WO 94/01483. As mentioned above, useful synthetic polymers include polyethylene, polypropylene, polyurethane, polyglycolic acid, polyesters, polyamides, their mixture, blends, copolymers, mixtures, blends and copolymers may be used, for example polyesters such as polyethylene terephthalate including DACRON (RTM) and MYLAR (RTM) and polyaramids such as KEVLAR (RTM), polyfluorocarbons such as polytetrafluoroethylene (PTFE) with and without copolymerized hexafluoropropylene, expanded or not-expanded PTFE, and porous or nonporous polyurethanes. Such materials include the expanded fluorocarbon polymers (especially PTFE) materials of GB 1355373, GB 1506432, GB 1506432 U.S. Pat. No. 3,953,566, U.S. Pat. No. 4,187,390, and U.S. Pat. No. 5,276,276.

Included in the class of useful fluoropolymers are polytetrafluoroethylene (PTFE), fluorinated ethylene propylene (FEP), copolymers of tetrafluorethylene (TFE), and perfluoro (propyl vinyl ether) (PFA), homopolymers of polychlorotrifluoroethylene (PCTFE), and its copolymers with TFE, ethylene-chlorotrifluoroethylene (ECTFE), copolymers of ethylene-tetrafluoroethylene (ETFE), polyvinylidene fluoride (PVDF), and polyvinyfluoride (PVF).

In addition, one or more radio-opaque metallic fibres, such as gold, platinum, platinum-tungsten, palladium, platinum-iridium, rhodium, tantalum, or alloys or composites of these metals may be incorporated into the grafts of the present invention, in order to allow fluoroscopic visualization of the graft.

Although the possibility of the auxetic tube being metallic is not excluded, production of the auxetic tube using a polymer of suitable tissue-compatibility is preferred since it eliminates the risk, which can occur where metallic tubes are deployed, of chemical reaction between the metal and its immediate environment. The use of relatively inert metals and alloys such as those discussed above may be preferred if the auxetic tube is to be metallic.

A tubular graft in accordance with the invention typically comprises a first auxetic tube produced by:
(a) machining appropriate geometry, e.g. inverted microhexagons, into the structure of a tube; or
(b) processing, i.e. compression and subsequent deformation of polymeric powder particles into a tubular form under controlled conditions of pressure and temperature; or
(c) a combination of processing and subsequent micromachining.

Appropriate geometry such as inverted microhexagons can be machined into the material which forms or is to form the first auxetic tube using an excimer laser system. Machining by means of excimer laser technology allows feature sizes from about 4 mm to about 2 microns to be etched into a wide variety of materials and features of the order of 10 microns in size or larger can be drilled through the entire thickness of a substrate. The structures detailed below in the specific embodiments of the invention have been manufactured using an excimer laser.

The tubular graft may be sufficiently flexible that, by virtue of the synclastic property of the auxetic first tube, it can be readily turned inside out within the confines of a duct, e.g. a blood vessel or other in vivo duct, in which it is to be installed or implanted, or from which it is intended to extend. For example, it may be desirable to have a graft of the present invention extending from a damaged duct and for there to be overlap between the damaged duct and the graft. This can be achieved by taking an inverted tubular graft of the present invention and inverting it in the damaged vessel so that the graft is in a non-inverted configuration. However, the ability to use the synclastic property of the auxetic tube is dependent upon the other components of the graft, namely the non-auxetic tubular covering on at least one of the interior and exterior surfaces. If the graft has a covering on its interior surface then by virtue of the fact that it is non-auxetic, any radial stretching of it during inversion of the graft will either result in a transverse shrinkage (i.e. a reduction in length along the longitudinal axis) (positive Poisson ratio materials) or no change in size along the longitudinal axis (zero Poisson ratio) whilst the auxetic first tube will expand along the longitudinal axis upon radial expansion.

Relevant prior art includes coronary stents made of, or based on, metal and are either self-expandable or capable of undergoing plastic deformation (i.e. they only deform when pressurised and cannot regain their original shape in the absence of an external force or pressure).

A wide range of vascular grafts are presently available including e.g. the Gore-Tex (RTM) Intering (RTM). A recognised problem encountered with grafts is that kinking can occur, as can over-compression. Prior art grafts seek to address these problems in various ways, but the issue of radial over-compression (which can result in blocking of the lumen of the graft) is one which has proved particularly problematic. Typically, one finds that in order to prevent blockage of a lumen, a tube is made with a thick wall, and this can be undesirable.

The present invention seeks to overcome these prior art disadvantages, and particularly to provide tubular grafts which provide increased counteraction upon compression, but whose longitudinal or localised radial flexibility is not inhibited or reduced under typical in vivo conditions as a result.

As mentioned above, one useful form of tubular graft of the present invention uses a geometry of inverted hexagons in order to effect auxetic properties in a tubular structure which would otherwise not be auxetic. These “inverted hexagons” are not “regular” hexagons and instead essentially comprise a hexagon having first and second sides opposite and parallel to one another, and then third, fourth, fifth and sixth inwardly-inclined sides joining them. The present inventor has found that by linking chains of such inverted hexagons together via their third, fourth, fifth and/or sixth sides, then an auxetic structure can be created. Obviously, it is possible to incorporate into such structures inverted hexagons which are linked together via the vertices of their first and second sides, although this may result in non-auxetic regions whilst still retaining the overall auxetic properties.

Since compression of the tubular liners is ultimately limited by the ability of the inverted hexagons to compress, there is as a result a maximum extent to which compression can be effected (i.e. the tubular liners have a minimum radius), and this is dictated by the construction of the inverted hexagons. As compression takes place, the tubular liner becomes more rigid in its structure at the point or region of compression and more resistant to deformation, the degree of which is controlled by the structure of the tubular liner (e.g. first and second sides perpendicular to the longitudinal axis, or parallel to it). For example, increasing the length of the connecting members increases the flexibility of the tubular liner.

The tubular grafts of the present invention can be structured to ensure that fluid flow can be achieved along their length by having a minimum radius to which they can be compressed. Prior art stents also fail to show the relative increase in strength upon compression achieved by the auxetic tubular liners of the present invention. In addition, the structures of the present invention can be made highly flexible, even when compressed.

The ability of the first auxetic tube to have a minimum diameter means that it can be particularly useful in situations where pressure may be exerted upon the graft which might (in conventional prior art grafts) result in blockage of the graft.

Thus in a tubular graft according to the present invention, defining a longitudinal axis between said first and second ends, the first auxetic tube can have a structure comprising a plurality of longitudinally elongate strips of interconnected hexagons oriented along said longitudinal axis, each longitudinally elongate strip comprising a plurality of interconnected hexagons having:
(i) first and second sides parallel with and opposite to one another;
(ii) third and fourth sides dependent from said first side; and
(iii) fifth and sixth sides dependent from said second side;
said third side being connected to said fifth side at a first vertex, and said fourth side being connected to said sixth side at a second vertex;
said first side of each hexagon making an internal angle of less than 90 degrees with each of said third and fourth sides, and said second side making an internal angle of less than 90 degrees with each of said fifth and sixth sides;
said first and second sides of said hexagons being oriented perpendicular to said longitudinal axis;
each hexagon being connected to at least a first adjacent hexagon, said first side of each hexagon comprising a second side of said first adjacent hexagon, and said second side comprising a first side of any second adjacent hexagon;
each longitudinally elongate strip being connected to first and second radially adjacent longitudinally elongate strips by a plurality of connecting members.

The plurality of connecting members may be between said third and fifth sides of said plurality of hexagons of said first adjacent radial loop and said fourth and sixth sides of said plurality of hexagons of said second adjacent radial loop.

The connecting members may be other than between the vertices of said first and second sides.

This orientation of structures (with strips as opposed to loops) is of particular use in the present invention since it allows the radial compressibility of the first auxetic tube of the graft to be reduced as compared to the radial compressibility achievable with an equivalent looped structure. This can be useful where it is important to ensure a relatively large minimum diameter for the graft in situ, as compared to use of auxetic structures in stents where greater compressibility is of use in accommodating e.g. vascular plaques without placing undue pressure on the vessel in which the stent has been placed.

In certain embodiments of the present invention, it may be desirable to arrange the adjacent loops of hexagons such that they are offset relative to one another. For example, it may be desirable to arrange a first loop so that the vertices of its first and second sides with its third and fifth sides are proximal to the vertices made between the fourth and sixth sides of hexagons of a second loop. For example, a connecting member may join the first and second loops by connecting the vertices of the first and second sides of the first loop (made with its third and fifth sides) to the vertex made between the fourth and sixth sides of the hexagons of the second loop.

Alternatively, the connecting members can for example be between said first vertex of said hexagons of said first loop and said second vertex of said hexagons of said second loop.

Examples of tubular grafts incorporating such first auxetic tubes are detailed below. Properties of the first auxetic tube, including the extent of its auxetic nature, can be modified depending upon the exact construction of the inverted hexagons. The above general structure is particularly useful where it is desired to have a first auxetic tube which is able to be expanded and compressed radially.

In particular, said connecting members-may be between said first vertex of said hexagons of said first loop and said second vertex of said hexagons of said second loop.

As well as the above first auxetic tube structures using inverted hexagons (in which the first and second parallel sides are oriented in the longitudinal axis of the tubular liner), structures can also be made in which the first and second parallel sides are oriented perpendicular to the longitudinal axis of the tubular liner. These structures whilst also being auxetic can be manufactured such that they are capable of little radial compression r expansion, yet are capable of substantial longitudinal compression or expansion.

Thus in a tubular graft according to the present invention, the first auxetic tube defining longitudinal axis between said first and second ends, said first auxetic tube may have a structure comprising a plurality of longitudinally elongate strips of interconnected hexagons oriented along said longitudinal axis, each longitudinally elongate strip comprising a plurality of interconnected hexagons having:
(i) first and second sides parallel with and opposite to one another;
(ii) third and fourth sides dependent from said first side; and
(iii) fifth and sixth sides dependent from said second side;
said third side being connected to said fifth side at a first vertex, and said fourth side being connected to said sixth side at a second vertex;
said first side of each hexagon making an internal angle of less than 90 degrees with each of said third and fourth sides, and said second side making an internal angle of less than 90 degrees with each of said fifth and sixth sides;
said first and second sides of said hexagons being oriented perpendicular to said longitudinal axis;
each hexagon being connected to at least a first adjacent hexagon, said first side of each hexagon comprising a second side of said first adjacent hexagon, and said second side comprising a first side of any second adjacent hexagon;
each longitudinally elongate strip being connected to first and second radially adjacent longitudinally elongate strips by a plurality of connecting members.

The plurality of connecting members may be between:
(a) said third and fifth sides of said plurality of hexagons of said longitudinally elongate strip and said fourth and sixth sides of said plurality of hexagons of said first radially adjacent longitudinally elongate strip; and
(b) said fourth and sixth sides of said plurality of hexagons of said longitudinally elongate strip and said third and fifth sides of said plurality of hexagons of said second radially adjacent longitudinally elongate strip.

The connecting members may be other than between the vertices of said first and second sides.

As for the looped arrangements of hexagons, the strips of hexagons may be offset relative to one-another and adjacent strips may be joined by connecting members appropriately.

In such a first auxetic tube, the connecting members may be between:
(a) said first vertex of said hexagons of a given longitudinally elongate strip and said second vertex of said hexagons of a first radially adjacent longitudinally elongate strip of hexagons; and
(b) said second vertex of said hexagons of said given longitudinally elongate strip and said first vertex of said hexagons of a second radially adjacent longitudinally elongate strip of hexagons.

In the various embodiments of the present invention which use polygons such as hexagons connected together forming either adjacent longitudinally elongate strips or adjacent radial loops, the connecting member can be shaped as desired, so long as the eventual structure defined is auxetic. For example, the connecting members can be straight, curved or angled.

The simplest possible shape is a straight one, and a straight connecting member can be arranged parallel to the first and second sides of a hexagon to which it is connected. As mentioned above, straight connectors can be between first and second vertices of adjacent hexagons, or they can be between e.g. third and fifth or fourth and sixth sides of adjacent hexagons. Alternatively, a straight connecting member can be arranged at an angle to the first and second sides of a hexagon to which it is connected.

Alternative structures include curved and angled structures. As mentioned above, the requirement is that the final structure incorporating the connecting members is auxetic. Therefore, in the above embodiments all of the hexagons cannot be connected by connecting members between vertices of first or second sides of adjacent hexagons.

As well as the above “inverted hexagon” structures, the use of other auxetic structures falls within the scope of the present invention. In particular, the first and second sides mentioned above which are parallel to and opposite one another can be replaced with e.g. sides having relatively inflexible branched sections. Thus for example first and second sides can be replaced with a first side having first and second vertices, and with first and second arms extending from each of the first and second vertices, each of the first and second arms making an internal angle with the first side of between 90 and 180 degrees. For example, internal angles of between 91 and 179 degrees can be made, e.g. 125, 130, 135, 140, 145 or 150 degrees. Third, fourth, fifth and sixth sides can then depend from the first and second arms of the first and second sides, thus completing the polygons. By making the third, fourth, fifth and sixth sides relatively flexible compared to the first and second sides and the first and second arms, the auxetic properties of the structures and tubular liners of the present invention are ensured. Examples of such structures are given below.

According to the present invention there is also provided a graft assembly comprising:
(i) a tubular graft according to the present invention;
(ii) a mandrel upon which said tubular graft is located; and
(iii) a sleeve surrounding said mandrel and tubular graft, said sleeve having an open end;
said mandrel being movable relative to said sleeve.

Also provided according to the present invention is the use of a tubular graft according to the present invention in the manufacture of an assembly according to the present invention.

The assembly and the tubular graft may be for use in duct repair (e.g. vascular repair).

Also provided according to the present invention is method of inserting a tubular graft according to the present invention into a duct, said tubular graft defining first and second faces, said first face facing said lumen, said second face facing away from said lumen, said method comprising the steps of:
(i) locating said tubular graft on a mandrel surrounded by a sleeve to define an assembly, said sleeve having an open end;
(ii) passing said assembly into said duct;
(iii) moving said mandrel relative to said sleeve so as to cause said tubular graft to be displaced through said sleeve open end such that said tubular graft folds back over said sleeve and inverts within the confines of said duct such that said second face faces said lumen of said inverted tubular liner and said first face faces away from said lumen of said inverted tubular graft;
(iv) withdrawing said sleeve and said mandrel from said duct, leaving said inverted tubular graft in situ.

The open end of the sleeve through which the graft is displaced may have a convexly curved end face to facilitate folding back of the graft offer the sleeve and pressure transduction in the lateral direction.

The mandrel may be provided with an ancillary element for use, for example, in softening up and/or pre-dilation of material deposited within the duct. Alternatively or additionally, the mandrel may be provided with a laser radiation transmission path, e.g. a fibre optic, to allow laser radiation to be directed into the duct, for instance to treat clogged or plaque-filled ducts.

The mandrel may define a leading end portion and be provided with a passageway or passageways in communication with said leading end portion of the mandrel to allow fluids to be withdrawn from the duct.

The first auxetic tube of the present invention can also be provided with polygonal shapes (such as “inverted hexagons”) of varying size.

As discussed above, in the case of hexagons (and also other polygons), different orientations of the polygons result in different properties for the tubular liner—in the case of hexagons, those having the first and second sides oriented in the longitudinal axis of the tubular liner are typically highly radially compressible compared to their longitudinal compressibility, and those with their first and second sides oriented perpendicular to the longitudinal axis of the tubular liner are highly longitudinally compressible compared to their radial compressibility.

The graft, particularly the first auxetic tube, may be used as a vehicle for delivery of drugs or other beneficial agents to a duct, particularly to sections of a duct adjacent the graft. In the case of a diseased vessel, wound-healing agents or DNA materials such as oligopeptides may be delivered from the graft. Such agents may be incorporated in the porous auxetic material, e.g. by chemical and/or physical fixation. The drug or other agent can be incorporated into the interstitial voids or it can be introduced by blending into polymeric particles which are to be used in production of the graft, for example by processing into a microporous auxetic first tube or into a non-auxetic tube which is subsequently transformed into an auxetic first tube, e.g. by micromachining, or the drug can be absorbed by, or adsorbed onto, a finished structure. Other uses of drugs are the coating of the outer and inner surfaces of the graft or the first auxetic tube. For example, the outer surface can be coated with a cell pacifier, whereas the inner (luminal) surface can be coated with an anticoagulant such as heparin. Alternatively, where a graft is provided comprising both exterior and interior coverings or where a graft is provided having more than one exterior covering or more than one interior covering, a layer of drugs may be provided between the coverings, for example in the form of a gel, allowing in vivo delivery of drugs to the patient.

As regards the inverted hexagons, it is mentioned above that they may vary in size in different parts of the first auxetic tube. The inverted hexagon structures can be sized in order that they facilitate the physical compliance of the graft when in vivo. For example, they inverted hexagons can be of a size greater than that given in the examples below. Similarly, the porosity and permeability of the non-auxetic tubular coverings can also be used to facilitate physical compliance of the graft.

The invention will be further apparent from the following description, with reference to the several figures of the accompanying drawings, which show, by way of example only, forms of grafts.

Of the Figures:

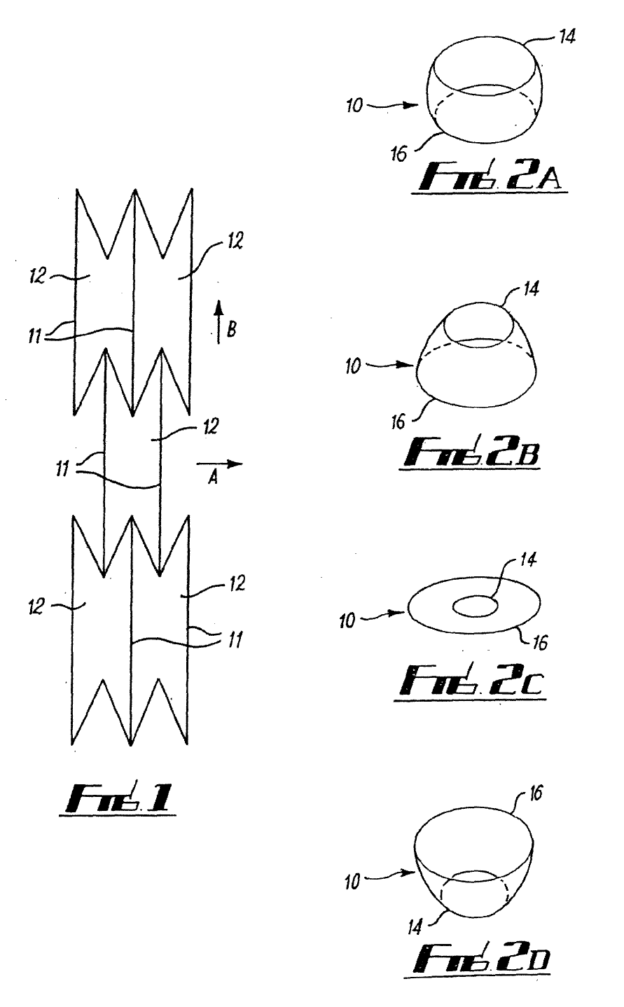

FIG. 1 shows the geometrical features of an auxetic material which may be made use of in a graft in accordance with the present invention;

FIG. 2 shows (FIGS. 2A to 2D) the inversion of an auxetic tubular structure of relatively short length;

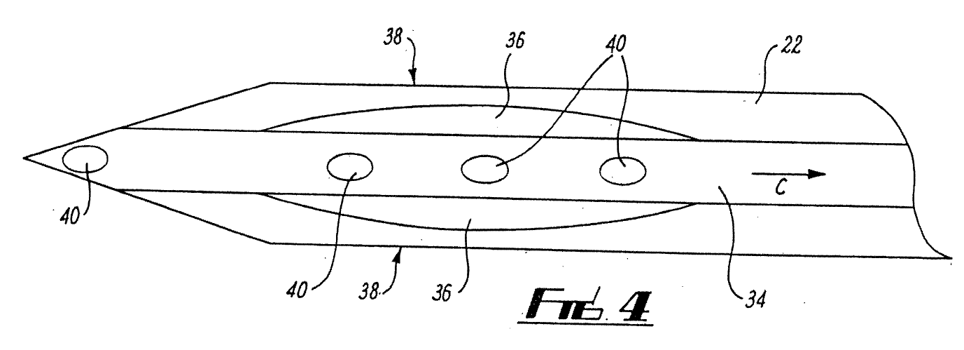

FIG. 3 shows a sectional view of an assembly for use in implanting a graft within an in vivo duct such as a blood vessel;

FIG. 4 shows an enlarged view showing details of the mandrel of the assembly shown in FIG. 3;

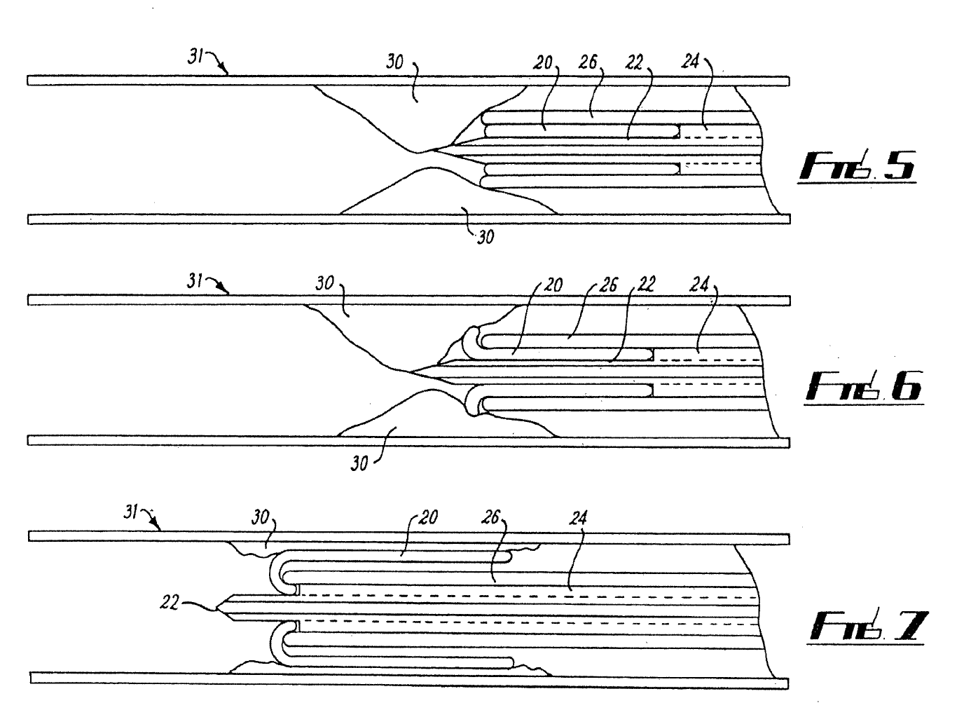

FIGS. 5-7 are views showing successive stages in the use of the assembly to implant the graft within a blood vessel or the like;

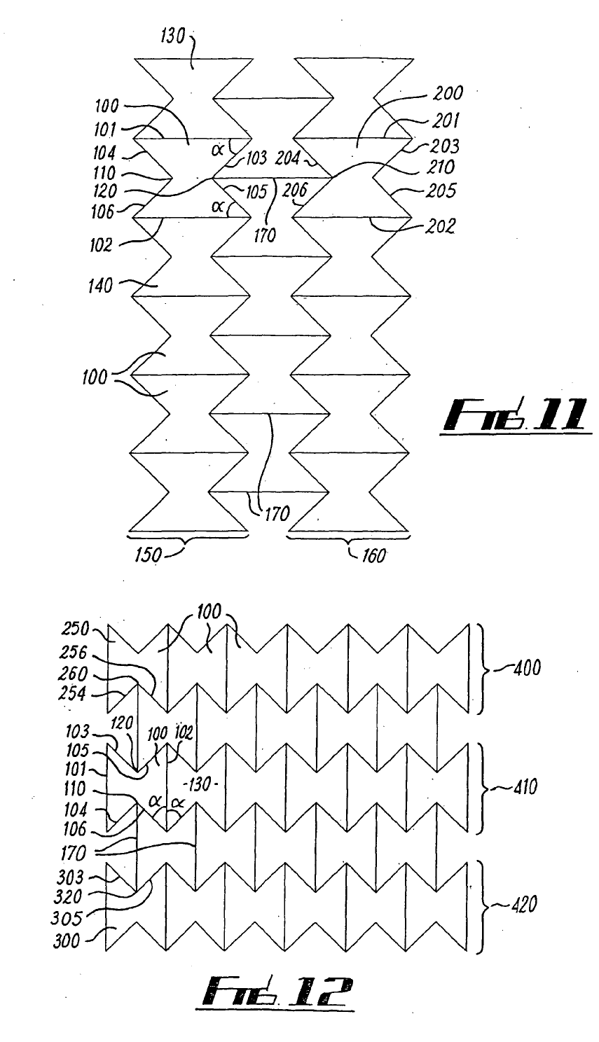

FIGS. 8-9 are views illustrating transfer of the graft on to the mandrel during the course of preparing the assembly of FIG. 3;

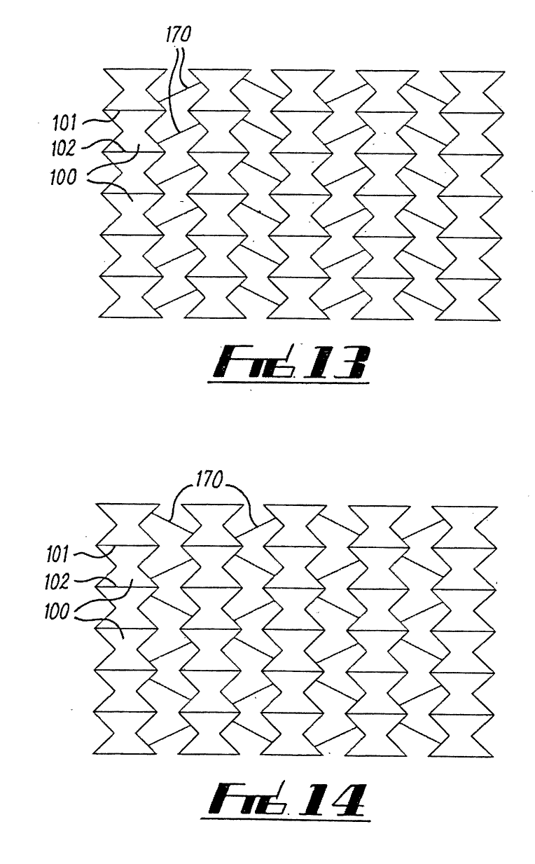

FIG. 10 shows the effect of compression of a section of auxetic tubular material;

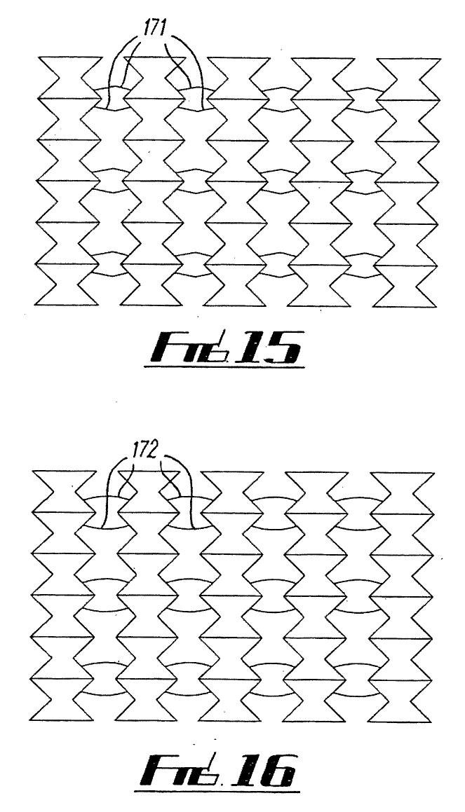

FIG. 11 shows a section of a first auxetic tube having an “inverted hexagon” structure.

FIG. 12 shows a section of a second auxetic tube having an “inverted hexagon” structure perpendicularly arranged relative to the structure of FIG. 11;

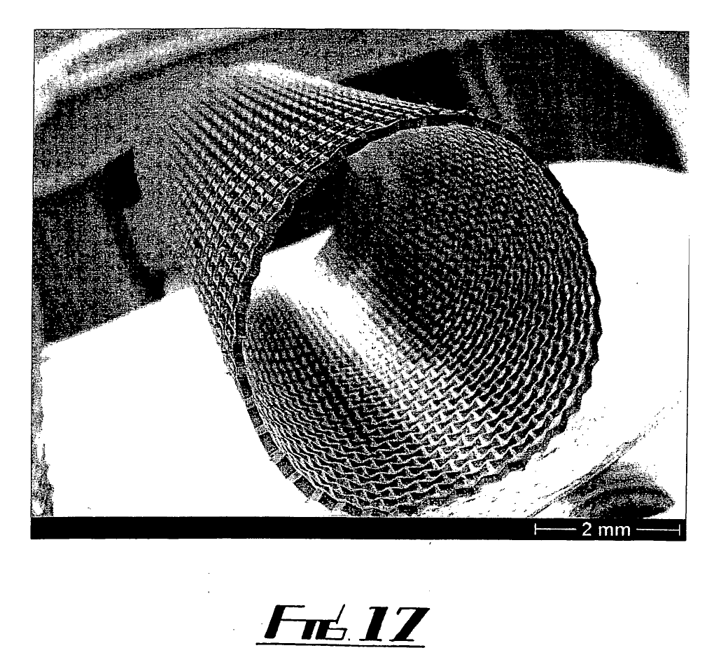

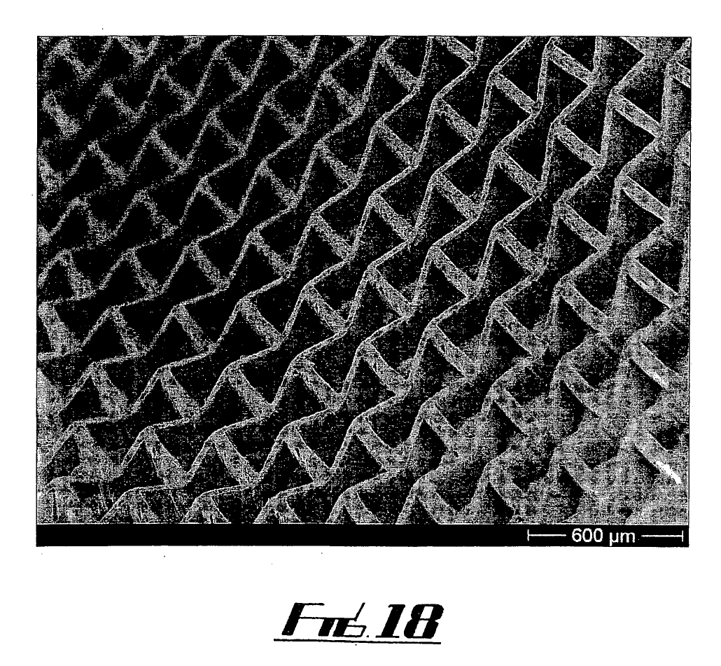

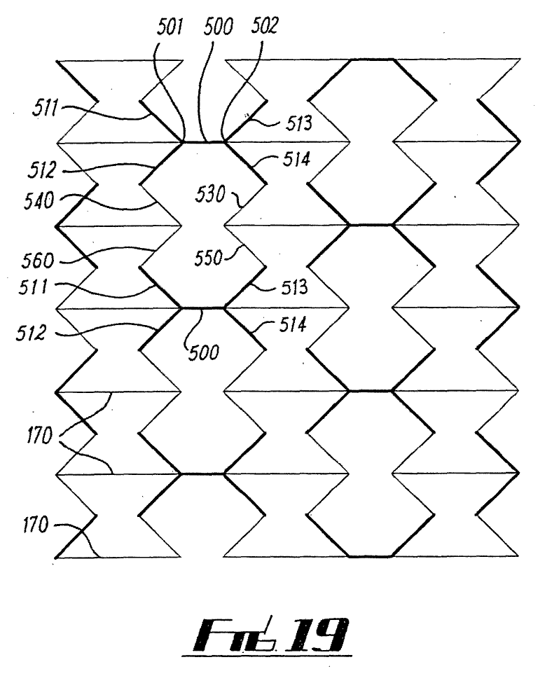

FIGS. 13-16 show alternative embodiments with an auxetic structure comprised of inverted hexagons and (FIGS. 13, 14) straight connecting members at an angle to the parallel first and second sides, angled connecting members (FIG. 15) and curved connecting members (FIG. 16);

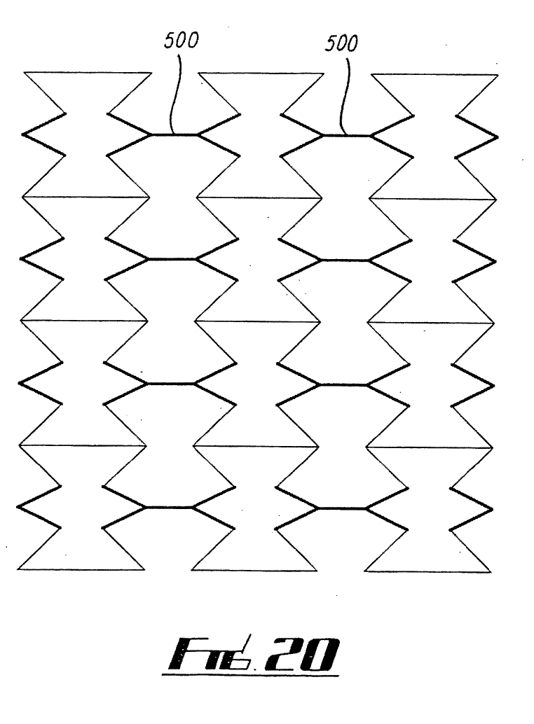

FIG. 17 shows a perspective view of a section of a first auxetic tube of the present invention having a diameter of about 6 mm and with hexagons having first and second sides (which are parallel with and opposite to one another) oriented in the longitudinal axis of the liner. Hexagons are approximately 613 μm in width and 471 μm in height. Wall thickness of the first auxetic tube is about 150 μm, and total length is about 2 cm;

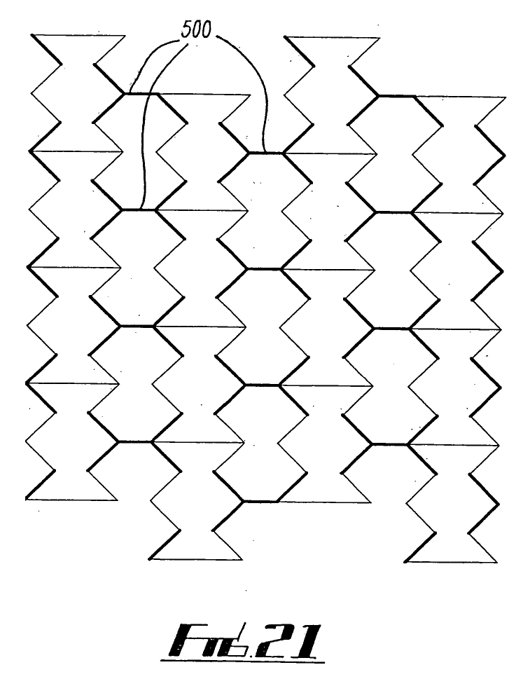

FIG. 18 shows a magnified view of the first auxetic tube of FIG. 17;

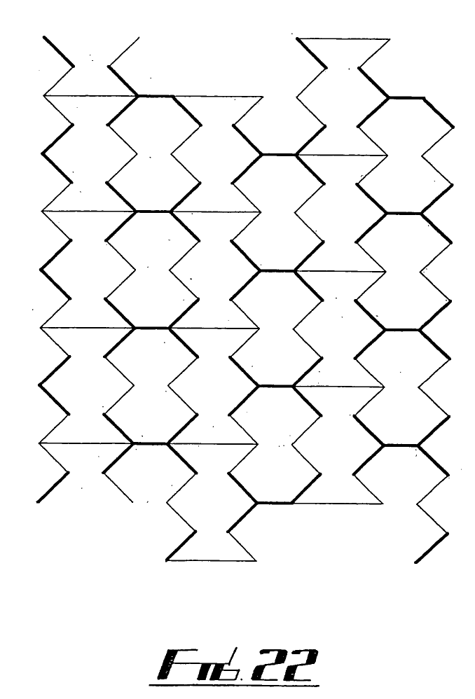

FIGS. 19-22 show alternative auxetic structures useful in the first auxetic tube of the present invention;

FIG. 23A shows a first graft having a first auxetic tube and an exterior non-auxetic covering;

FIG. 23B shows a first graft having a first auxetic tube and an interior non-auxetic covering; and

FIG. 23B shows a first graft having a first auxetic tube, an exterior non-auxetic covering, and an interior non-auxetic covering.

The grafts of the present invention are shown in FIGS. 23A-C and their characteristics and construction of the first auxetic tube are described in detail subsequently.

As can be seen from FIG. 23A, a graft 20 is formed from a first auxetic tube 20D and an exterior non-auxetic covering 20A. In one embodiment, auxetic tube 20D is compressed and inserted into the lumen of non-auxetic covering 20A and expanded such that it is contiguous with non-auxetic covering 20A. In another embodiment, auxetic tube 20D has a non-auxetic covering 20A spun around it.

As can be seen from FIG. 23B, a graft 20 is formed from a first auxetic tube 20D and an interior non-auxetic covering 20B. Auxetic tube 20D and non-auxetic interior covering 20B are co-extruded and the interface between the two layers of material is then laser-treated in order to join the layers such that the non-auxetic interior covering 20B is secured to the first auxetic tube 20D.

In a third embodiment shown in FIG. 23C, a graft 20 is formed from a first auxetic tube 20D, an interior non-auxetic covering 20B and an exterior non-auxetic covering 20A. The graft is manufactured by firstly following the steps described above for the manufacture of the graft 20B. The product of that process is then put through the steps described for the manufacture of the graft 20A, giving the final graft 20.

With regard to auxetic structures used in the grafts of the present invention, referring to FIG. 1, this illustrates a typical geometry (inverted hexagons 12 or bow tie honeycomb) which may be micromachined by for example excimer laser technology so as to impart auxetic properties to a substrate material. It will be seen that the application of a tensile load in direction A will result in expansion of the structure in direction B in contrast with conventional materials having a positive Poisson ratio. However, the present invention is not limited to securing auxetic properties by micromachining of geometrical features; such properties may be derived by other methods known in the art, e.g. compression and deformation of polymeric powder particles into a tubular structure under controlled temperature and pressure conditions to produce a material which is, in effect, intrinsically auxetic.

Consideration of the synclastic property of auxetic materials has led the present applicant to the recognition that a graft, e.g. a stent for implantation in an in vivo duct, may be readily inverted or turned inside out. Expansion and inversion of a compressed stent initially retained between a mandrel and sleeve results in the release of energy into the plaque (or other blockage) when it is contacted by the inverted stent, thus effecting e.g. dilation of the plaque. This effect is illustrated in FIGS. 2A to 2D. Starting with a relatively short section of a tubular structure 10 having upper and lower ends 14, 16 (FIG. 2A), the structure is compressed laterally, which for the purposes of illustration is supported by a surface underneath its lower surface 16. The structure may be manipulated by releasing the lower end 16, whose diameter as a result increases, while at the same time pressing the upper end 14 towards the support structure (FIG. 2B). For example, this effect is possible if the structure 10 is based on the inverted microhexagon geometry of FIG. 1 so arranged that the sides 11 of the hexagons (i.e. the first and second sides of a hexagon which are parallel with and opposite to one another) are oriented in the circumferential direction with respect to the structure 10, i.e. are oriented perpendicular to the longitudinal axis of the graft. A similar effect can be achieved with hexagons whose first and second sides (which are parallel with and opposite to one another) are oriented in the longitudinal axis of the graft.

Assuming that the material forming the structure 10 is sufficiently flexible, such compression may be continued until the upper end 14 is drawn towards the plane containing the lower end 16 (see FIG. 2C) thus allowing it to be passed through that plane so that, as shown in FIG. 2D, the tubular structure is inverted or turned inside out and the upper end 14 becomes the lower end 16 and vice versa.

The above inversion effect is exploited in the present invention for the purpose of lining a duct, e.g. inserting a stent into an obstructed or narrowed duct in that the liner or stent employed is of an auxetic material and is sufficiently flexible that it may be inverted within the confines of the duct.

Referring now to FIGS. 3 to 7, graft 20 comprises a first auxetic tube of material which may be intrinsically auxetic or may have been rendered auxetic by suitable techniques such as micromachining of appropriate geometrical features. The graft 20 is located on a reduced diameter leading portion 22 of a mandrel 24 and is in a compressed state between the portion 22 and an outer sleeve 26. The mandrel 24 and the sleeve 26 are arranged so as to be movable relative to one another and are typically made of a low friction/non-stick material such as polytetrafluoroethylene.

The tip 28 of the mandrel portion 22 is of tapering configuration and initially projects to some extent beyond the leading end of the sleeve 26. The assembly comprising the mandrel, graft and sleeve is, in use, coupled to a catheter device so that it can be introduced in the usual manner and positioned in the vicinity of an obstruction or narrowing of the blood vessel. The arrangement is such that the user may operate the assembly through the catheter device to effect movement of the mandrel 24 relative to the sleeve 26 as desired.

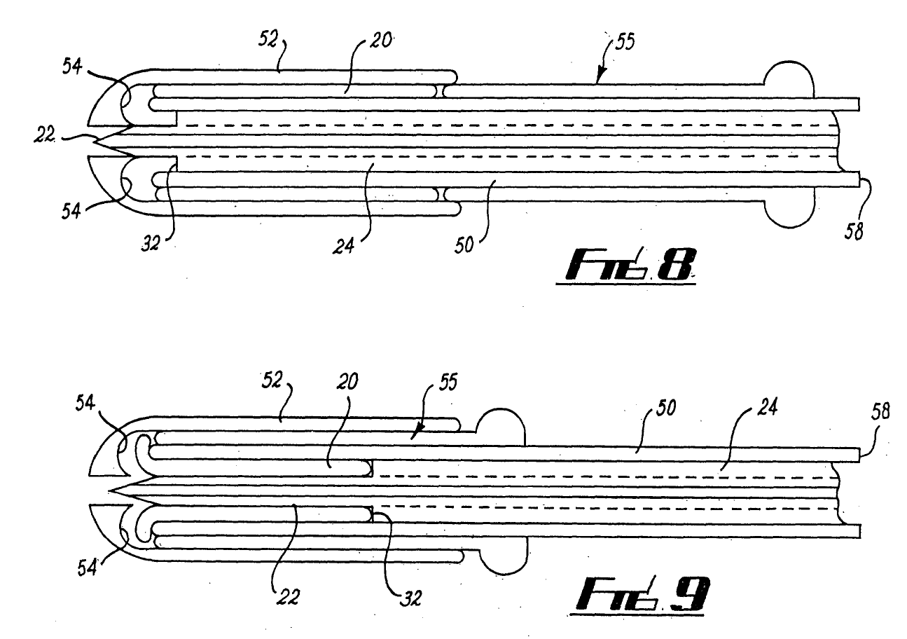

Initially or at some point during the procedure, the leading end of the graft 20 projects beyond the leading end of the sleeve 26 and by virtue of its auxetic properties tends to curl around that end in the manner illustrated in FIG. 6. To facilitate this, the end face 29 of the sleeve 26 is convexly curved.

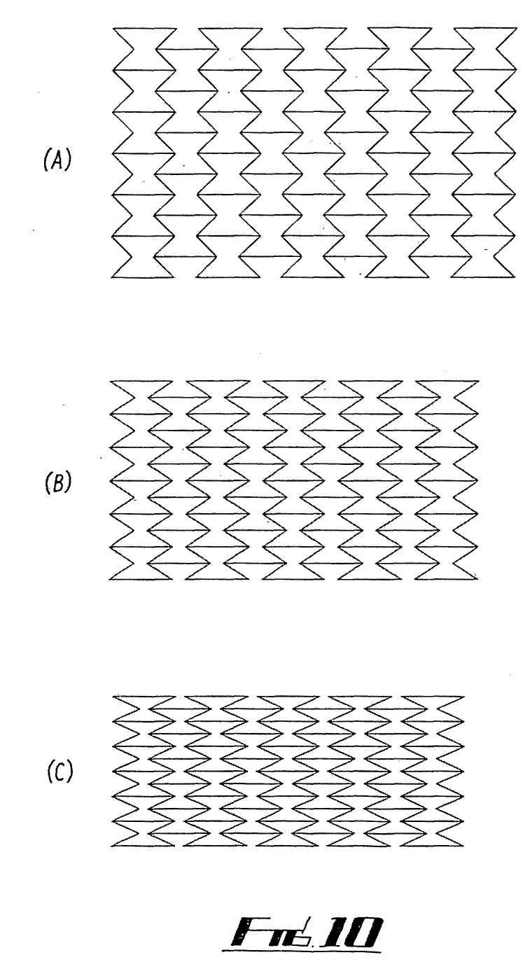

Once the assembly has been positioned close to the site of obstruction or narrowing of the duct 31 (see plaque deposits 30 in FIGS. 5 to 7) with the aid of a catheter, the mandrel 24 can be manipulated to move forwardly relative to the sleeve 26 so that the graft 20 is advanced forwardly also through its contact with shoulder 32 at the junction between mandrel portion 22 and the remainder of the mandrel. By progressive manipulative operations of the mandrel and sleeve, the graft 20 can be caused to begin inverting so that it folds back over the exterior of the sleeve 26. At the same time, as the graft passes out of the gap between the mandrel portion 22 and the sleeve 26, it is no longer subjected to compression and because of its auxetic properties, it can expand and exert lateral pressure so as to dilate the vessel. In this manner, the graft can be transferred from the assembly into the blood vessel and expand and exert pressure on the plaque or deposit to reduce the obstruction or narrowing (see FIG. 7). Eventually after the graft 20 has been fully deployed within the blood vessel, the mandrel 24 and sleeve 26 may be withdrawn with the aid of the catheter leaving the graft in situ.

Upon self-expansion, the graft forms a region of relatively high curvature during the time that it is undergoing inversion. The resulting “travelling” curved front affords the potential for exerting a sufficiently high pressure to flatten any lesion or further flatten it after pre-dilation.

To facilitate pre-dilation of the duct and thereby assist lining up of the graft during deployment, the mandrel 24 may be designed so that, in the region of its leading end, it may be radially expanded. This can be implemented by providing the mandrel with a central rod 34 which extends through a longitudinal passageway in the mandrel and which has its leading end captive with the leading end of the mandrel portion 22. A section 38 of the portion 22 is formed with a cavity 36 (see FIG. 4) and the walls of the portion 22 is provided with a number of longitudinal slits or apertures (not illustrated) so that this section 38 of the portion 22 can be caused to expand radially by pulling the rod 34 backwards in direction C relative to mandrel 24. When the mandrel is displaced forwardly of the sleeve 26 so as to expose the slitted or apertured section 38, expansion of the section 38 can be effected by manipulation of the rod 34 and mandrel 24 and this can be used to pre-dilate the deposit or plaque 30 to some extent in the artery or duct. One form of rod 34 is a quartz fibre optic catheter through which radiation, e.g. near-ultraviolet radiation from an excimer laser, may be transmitted to the leading end of the mandrel to treat the deposit or plaque material obstructing the artery or the like.

Another feature that may be employed is to provide the mandrel with a longitudinal passageway through which fluidised material (e.g. created by heating or laser treatment of the deposit) can be withdrawn or through which blood flow can be facilitated during graft deployment. In the embodiment illustrated in FIG. 4, this is implemented by using a hollow rod 34 having holes 40 at its distal end to allow fluid entry into the passageway within the rod. Some of the holes may be provided in registry with the cavity 36 so that fluidised material entering via the longitudinal slits or apertures of section 3 8 can be drawn into the interior of the hollow rod 34.

In a modification as illustrated in FIG. 3 by phantom lines the mandrel 24 may be telescopic with the portion 22 forming an inner section 22A telescopically received within an outer section 24A of the mandrel so that the inner and outer mandrel sections can be displaced relative to one another when it is convenient to do so, e.g. during graft deployment or during fabrication of the assembly comprising the graft, mandrel and sleeve (as described below with reference to FIGS. 8 and 9). This arrangement may for instance be employed, in conjunction with the expansion feature described with reference to FIG. 4, to facilitate back-folding of the initial part of the graft around the leading end of the sleeve 26.

In another modification, as discussed hereinbefore, a pathway or pathways may be provided for fluid flow from one end of the assembly to the other so that, for example, blood may flow through the assembly from a location upstream of the narrowing or obstruction in an artery to a location downstream thereof The fluid flow pathway(s) may for instance be provided by the provision of strategically located apertures or slits in the sleeve 26 and the mandrel 22, 24.

Referring now to FIGS. 8 and 9, the production of the assembly comprising the compressed graft 20, the mandrel 24 and the sleeve 26 is illustrated. Initially the graft 20 of auxetic material is manufactured around a tubular former 50 which is assembled with the mandrel 24 and a housing 52. The housing 52 functions in extruder-like fashion and has an internal curved end face 54 acting as a guide for transfer of the auxetic tube from the former 50 onto the mandrel portion 22. A plunger 55 is assembled to the former 50 (see FIG. 8) and is advanced forwardly to displace the graft 20 and “extrude” it out of the gap between the former 50 and the housing 52 and onto the mandrel portion 22 (see FIG. 9). At the same time, the mandrel 22 is displaced so that the graft 20 locates on to the mandrel section 22 with one end of the graft 20 immediately adjacent the shoulder 32. Once the graft 20 has been transferred to the mandrel, the housing 52 may be removed and the sleeve 26 is used to displace the former 50 by abutting the leading end of the sleeve 26 against the trailing end 58 of the former and moving the sleeve 26 forwardly to slide the former 50 over the graft 20 until the sleeve 26 is substituted for the former 50. In this way, the auxetic tube forming the graft 20 is located, in a compressed state, between the mandrel portion 22 and the sleeve 26.

It is envisaged that the double curvature property of auxetic materials will confer advantages relative to conventional metal or metal-based grafts in that graft removal by mechanical manipulation may be facilitated without damaging the surrounding artery.

The auxetic nature of the first auxetic tubes of the grafts of the present invention is shown in FIG. 10, which shows sections of a first auxetic tube of the present invention. The sides of the hexagons at (A), (B) and (C) remain the same length. Vertical (radial) compression effects an approximately 13% longitudinal compression and an approximately 40% circumferential compression comparing (A) to (C), equating to an approximate 64% radial compression. The general nature of auxetic structures (as used in the present invention) means that compressing the graft radially will cause a longitudinal compression (shortening). Similarly, a longitudinal expansion (lengthening) will cause a radial expansion. This ability to compress and expand means that the grafts of the present invention are also highly flexible, and expansion of a graft which also causes longitudinal expansion can aid in effecting an inversion of the graft (so long as the graft to be inverted is not oriented with a non-auxetic interior covering (although of course it can have a non-auxetic exterior covering which upon inversion will form a non-auxetic interior covering).

FIG. 11 shows a section of a first auxetic tube having first and second ends (not shown) defining a longitudinal axis between them, and having a first inverted hexagon structure comprising a plurality on inverted hexagons 110. Each hexagon 100 has: first and second sides 101,102 parallel with and opposite one another; third and fourth sides 103,104 depending from first side 101; fifth and sixth sides 105,106 depending from second side 102. Fourth side 104 is connected to sixth side 106 at second vertex 110, and third side 13 is connected to fifth side 105 at first vertex 120. First side 101 of each hexagon 100 makes an internal angle alpha of less than 90 degrees with each of sides 103,104 and, and second side 102 of each hexagon 100 makes an internal angle alpha of less than 90 degrees with each of sides 105,106.

Sides 101,102 are oriented in the longitudinal axis of the tubular liner.

Each hexagon 100 is connected to first and second adjacent hexagons. Thus for example first side 101 of hexagon 100 comprises a second side of first adjacent hexagon 130, and second side 102 comprises a first side of second adjacent hexagon 140.

The connected hexagons define radial loops 150,160 of interconnected hexagons, the adjacent radial loops being connected by a plurality of connecting members 170.

The exact orientation and arrangement (i.e. positioning) of the connecting members 170 varies between different embodiments of the invention. In this one, a connecting member 170 connects hexagon 100 with hexagon 200 having first and second sides 201,202 parallel with and opposite to one another, third and fourth sides 203,204 depending from first side 201, and fifth and sixth sides 205,206 depending from second side 202. Fourth side 204 is connected to sixth side 206 at second vertex 210.

Connecting member 170 connects hexagons 100,200 between first vertex 120 and second vertex 210.

Each of sides 101,102 is approximately 41 μm wide. The distance between sides 101, 102 is approximately 430 μm. Sides 101,102 are approximately 613 μm in length. Sides 103-106 are approximately 30 μm wide, hence their flexibility relative to sides 101,102. The distance between vertices 110,120 is approximately 118 μm. Angle alpha is approximately 46.85 degrees. There are a total of 40 hexagons 100 per circumference of the tubular liner.

Variation in thickness of the tubular liner can be used to e.g. modify its flexibility.

Such inverted hexagon structures provide additional advantages over prior art vascular grafts. In particular, the tubular liner of the present invention may act as an embolic containment device, helping to prevent the release of embolic particles into the bloodstream which is a high risk with balloon angioplasty.

In FIG. 12, the same general structure as shown in FIG. 11 is used, albeit oriented perpendicularly to the longitudinal axis of the tubular liner.

Thus, FIG. 12 shows a section of a first auxetic tube having first and second ends (not shown) defining a longitudinal axis between them, and having a first inverted hexagon structure comprising a plurality on inverted hexagons 100. Each hexagon 100 has: first and second sides 101,102 parallel with and opposite one another; third and fourth sides 103,104 depending from first side 101; fifth and sixth sides 105,106 depending from second side 102. Fourth side 104 is connected to sixth side 106 at second vertex 110, and third side 13 is connected to fifth side 105 at first vertex 120. First side 101 of each hexagon 100 makes an internal angle alpha of less than 90 degrees with each of sides 103,104 and, and second side 102 of each hexagon 100 makes an internal angle alpha of less than 90 degrees with each of sides 105,106.

Sides 101,102 are oriented perpendicular to the longitudinal axis of the tubular liner.

Each hexagon is connected to at least a first adjacent hexagon. Thus first side of hexagon 130 is connected to second side 102 of hexagon 100. Hexagons 100 define longitudinally elongate strips 400,410,420. Thus longitudinally elongate strip 410 is connected to first and second radially adjacent longitudinally elongate strips 400,420 by a plurality of connecting members 170. As mentioned above, the orientation and positioning of connecting members varies between different embodiments of the invention.

In the case of the auxetic tubular liners of FIG. 12, hexagon 250 of radially adjacent strip 400 comprises fourth and sixth sides 254,256 joined at vertex 260. Hexagon 300 of radially adjacent strip 420 comprises fourth and sixth sides 304,306 joined at vertex 320. Connecting member 170 joins vertex 120 to vertex 160, and another connecting member 170 joins vertex 110 to vertex 320.

In both of the above cases, connecting members 170 are parallel to the first and second sides. In other embodiments shown in FIGS. 13 and 14, different arrangements of straight connecting members 170 are shown. In FIGS. 15 and 16, non-straight connecting members are used. Specifically, connecting members 171 are angled, and connecting members 172 are curved.

In the case of non-straight connecting members which are capable of flexing in response to force exerted upon them, in order for the structure of the tubular liner to be auxetic then the flexing of the connecting members must not be such that it results in non-auxetic properties. For example, an angled connecting member with a large total length (for example having a single vertex with a small angle) and which is highly flexible could deform upon the exertion of pressure such that the structure was not auxetic. Conversely, an angled connecting member with a shorter total length, and which is much less flexible (possibly having a single vertex making a larger angle) will be less flexible and therefore the structure may remain auxetic. The same basic principle also applies to other non-straight connecting member shapes (e.g. curves).

In certain embodiments of the present invention, the adjacent loops of hexagons are arranged such that they are offset relative to one another, e.g. with a first loop arranged so that the vertices of its first and second sides with its third and fifth sides are proximal to the vertices made between the fourth and sixth sides of hexagons of a second loop (or adjacent strip) of hexagons.

FIGS. 17 and 18 show auxetic tubular liners made according to the present invention, and which are capable of being inverted e.g. using a mandrel as described above. The tubular liners are fabricated from nylon tubing (although other materials such as e.g. polyurethanes and others as discussed above can be used) made by taking a tube and placing a mask over a section of the tube, the mask having a structure cut into it which is a negative of the desired structure of the tubular liner. An excimer laser is then used to etch (ablate) the pattern defined by the mask from the tube, thus leaving a section of the desired auxetic structure. The mask is then moved and the process repeated to extend the pattern etched from the tube and produce a first auxetic tube having a desired structure. A wide range of parameters for the excimer laser are available, for example energy density and frequency, and focal length. Other parameters such as mask size, ablation ratio, and material of the tube can also be altered in order to achieve optimum results. In some cases the generation of plasma by a laser beam impacting a surface being etched results in small “rests” being left on the resulting structure. An ultrasonic bath can aid in the removal of any “rests”, should that be necessary or desired.

Generally, since the excimer laser is used to cut the auxetic structure into the e.g. polyurethane materials, the auxetic structure has a predefined or natural set of dimensions to which it will tend. It can, of course, be expanded or compressed, and also inverted. However, it will always tend back to the original dimensions of the tubular liner material.

In addition, certain embodiments of the present invention have first and second sides parallel with and opposite to one another replaced with thick sides having relatively inflexible thick branched sections extending from them. In such cases, the resulting polygons can still be considered to be the above “hexagons”, albeit with their first and second sides replaced with structures which although not straight do not detract from the auxetic nature of the structure. Importantly, the third, fourth, fifth and sixth sides remain flexible such that they can modify their conformation/shape and effect auxetic properties for the tubular liner.

As is shown in FIG. 19, first and second sides are replaced with a first side 500 having first and second vertices 501,502, and with first and second arms 511,512 extending from vertex 501 and arms 513,514 extending from second vertex 502, each of first and second arms 501-504 making an internal angle with first side 500 of between 90 and 180 degrees (in the case shown, approximately 135 degrees). Third, fourth, fifth and sixth sides 530,540,550,560 depend from the first and second arms of the first and second sides, thus completing the polygons. Sides 530-560 are relatively flexible compared to the first and second sides 500 and arms 511-514, ensuring the auxetic properties of the structures and tubular liners.

FIG. 19 also shows that it is possible for connecting members 170 to connect vertices of the first and second sides 500 with e.g. vertices made between third and fifth sides, or fourth and sixth sides, and for the resulting structure to be auxetic. Notably, there is no connection of a first or second side with an adjacent first or second side of an adjacent hexagon/polygon.

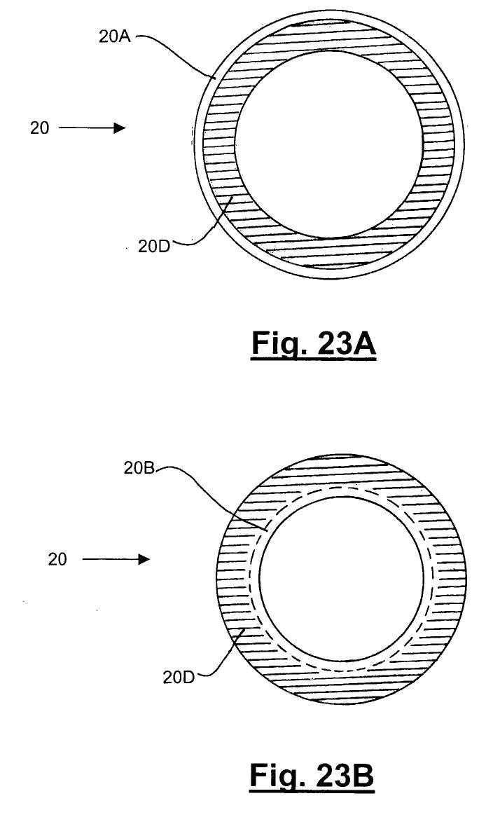

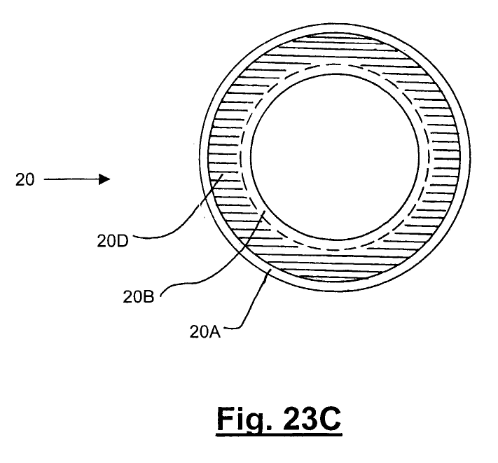

The structures shown in FIGS. 20-22 are also auxetic, and can also be used in the present invention. As is shown in FIGS. 21 and 22, structures which have connecting members joined to the vertices of the first and second sides can be auxetic, in this case joining the vertices of the first and second sides to the vertices between the third and fifth and fourth and sixth sides. The structure shown in FIG. 21 is more auxetic than that shown in FIG. 2 since a greater proportion of the connecting members 170 are able to move relative to the first and second sides of adjacent hexagons.

Whilst endeavouring in the foregoing specification to draw attention to those features of the invention believed to be of particular importance, it should be understood that the applicant claims protection in respect of any patentable feature or combination of features disclosed herein and/or shown in the drawings whether or not particular emphasis has been placed on such feature or features.

1-11. (canceled)

12. A tandem pressing apparatus comprising:
a tandem pressing line comprising a plurality of tandem presses disposed side by side; and
a work conveying apparatus for conveying a work (W) between the adjacent tandem presses;
wherein each of the tandem presses of the tandem pressing line includes a bed, plural uprights studded on the bed, and a slide supported on the uprights to be ascended or descended;
wherein the work conveying apparatus includes a main member and an arm member, the main member provided at a portion located inside the uprights of an adjacent pair of tandem presses of the tandem pressing line, and not interfering with the slide; and
wherein the arm member is movable between a position to enter into and retract from an upstream tandem press, and a position to enter into and retract from a downstream tandem press, for transferring the work from the upstream tandem press to the downstream tandem press.

13. A tandem pressing apparatus according to claim 12, wherein the main member is disposed in a space formed between an upright of the upstream tandem press and an upright of the downstream tandem press adjacent to the upstream tandem press, and including a space existing inside the upstream upright and the downstream upright.

14. A tandem pressing apparatus according to claim 13, wherein the main member is positioned outside a contour of the slide.

15. A tandem pressing apparatus according to claim 14, wherein the main member is fixed to the upright located at one side relative to a conveying direction of the work.

16. A tandem pressing apparatus according to claim 12, wherein the main member is slidably held by a guiding member provided inside the upright of the upstream tandem press and the upright of the downstream tandem press.

17. A tandem pressing apparatus according to claim 16, wherein the main member, moved to the upstream tandem press or the downstream tandem press, is positioned outside a contour of the slide.

18. A tandem pressing apparatus according to claim 17, wherein the guiding member is fixed to the uprights located at both sides of the slide in a direction generally orthogonal to the conveying direction of the work.

19. A tandem pressing apparatus according to claim 13, wherein the arm member is a multi-joint arm including two or more joints.

20. A tandem pressing apparatus according to claim 13, wherein the main member is fixed to at an intermediate portion of the upright in the height direction, and the arm member is extended laterally from the main member.

21. A tandem pressing apparatus according to claim 16, wherein the guiding member is fixed to at an intermediate portion of the upright in the height direction, and the arm member is extended downwardly from the main member.

22. A tandem pressing apparatus according to claim 13, wherein said work conveying apparatus is a conveying robot controlled by a CPU.

23. A tandem pressing apparatus according to claim 16, wherein the arm member is a multi-joint arm including two or more joints.

24. A tandem pressing apparatus according to claim 16, wherein said work conveying apparatus is a conveying robot controlled by a CPU.

25. A method of pressing a work, comprising:
providing a tandem pressing apparatus, said apparatus comprising a tandem pressing line comprising: a plurality of tandem presses disposed adjacent one another; and a conveying apparatus for conveying the work between said adjacent tandem presses; wherein each of the tandem presses comprises a bed, plural uprights, studded on the bed, and a slide supported on the uprights to be ascended or descended; wherein the work conveying apparatus comprises a main member and an arm member, said main member provided at a position located inside the uprights of an adjacent pair of tandem presses, and not interfering with the slide; and wherein the arm member is movable between a first position to enter into and retract from an upstream tandem press, and a second position to enter into and retract from a downstream tandem press, for transferring the work from the upstream tandem press to the downstream tandem press;
moving the arm member into the upstream tandem press when the work ascends the slide of the upstream tandem press;
catching the work with the arm member;
moving the arm member and the work out of the upstream tandem press;
moving the arm member and the work to the slide of the downstream tandem press;
leaving the work within the downstream tandem press; and
moving the arm member out of the downstream tandem press.

26. A method of pressing a work, comprising:
providing a processing apparatus, comprising upstream and downstream presses disposed adjacent one another, and a conveying apparatus conveying the work between the upstream press and the downstream press, each of said presses comprising a slide for receiving the work, and said work conveying apparatus comprising a main member and an arm member, said main member provided at a position not interfering with the slide, and said arm member being movable between a first position entering into and retracting from the upstream press, and a second position entering into and retracting from the downstream press, to thereby transfer the work from the upstream press to the downstream press;
moving the arm to the first position when the work is on the slide of the upstream press;
acquiring the work with the arm;
moving the work to the downstream press; and
depositing the work on the downstream press.
